(19) United States
(12) Patent Application Publication
(10) Pub. No.: US 2017/0056647 A1
(43) Pub. Date: Mar. 2, 2017

(54) SYSTEMS AND METHODS FOR ATTACHING FLEXIBLE CONDUCTIVE FILAMENTS, NETWORKS, OR PATCHES TO TISSUE VIA CATHETER DELIVERY

(71) Applicants: WILLIAM MARSH RICE UNIVERSITY, Houston, TX (US); BAYLOR COLLEGE OF MEDICINE, Houston, TX (US); TEXAS HEART INSTITUTE, Houston, TX (US)

(72) Inventors: Flavia Vitale, Houston, TX (US); Dimitri Tsentalovich, Houston, TX (US); Francesca Mirri, Houston, TX (US); Matteo Pasquali, Houston, TX (US); Mehdi Razavi, Houston, TX (US); Mark D. McCauley, Houston, TX (US); Colin C. Young, Houston, TX (US)

(73) Assignees: William Marsh Rice University, Houston, TX (US); Baylor College of Medicine, Houston, TX (US); Texas Heart Institute, Houston, TX (US)

(21) Appl. No.: 15/120,291

(22) PCT Filed: Feb. 20, 2015
(86) PCT No.: PCT/US2015/016869

§ 371 Date: Aug. 19, 2016

Related U.S. Application Data
(60) Provisional application No. 61/942,223, filed on Feb. 20, 2014.

(51) Int. Cl. A61N 1/05

(52) U.S. Cl. CPC A61N 1/0573; A61N 2001/058

(57) ABSTRACT

Systems and methods for deploying and securing conductive materials to a region of tissue may utilize a catheter. The catheter may provide a tip with one or more detachable sections or may provide an adjustable opening. A lumen of the catheter may provide a conductive material, such as a filament, fiber, network or patch of carbon nanotubes (CNTs) or carbon nanofibers (CNFs). In some embodiments, the conductive materials may be coupled to securing mechanisms, such as screws, clips, anchors, alligator clips, or anchors with barbs, which can be actuated to attach the conductive materials to desired regions of tissue. In some embodiments, the catheter may provide a needle tip that allows the conductive material to be embedded into desired regions of tissue by inserting the needle into the tissue.

### RELATED APPLICATIONS

This application claims the benefit of U.S. Provisional Patent Application No. 61/942,223, filed on Feb. 20, 2014, which is incorporated herein by reference.

### FIELD OF THE INVENTION

This invention relates to systems and methods for attaching flexible conductive filaments, networks, or patches to tissue via catheter delivery. More particularly, to attaching flexible conductive filaments, networks, or patches of carbon nanotubes to tissue with decreased or null conduction.

### BACKGROUND OF INVENTION

Methods of treating cardiac arrhythmia were discussed in PCT/US14/55893, filed on Sep. 16, 2014, which is incorporated by reference herein. As discussed in PCT/US14/55893, impaired regions of tissue may result in potential health issues. For example, regions of the myocardium with poor or null electric conductivity (e.g. scar tissue) may interfere with normal function of the heart, thereby leading arrhythmia or dangerous disruption of proper cardiac function. As noted in PCT/US14/55893, an impaired region of tissue may be improved by applying an electrically conductive material across the impaired region.

Systems and method for attaching flexible conductive filaments, networks, or patches to tissue via catheter delivery are discussed herein.

### SUMMARY OF INVENTION

In one embodiment, systems and methods for deploying and securing conductive materials to a region of tissue may utilize a catheter with a tip of one or more detachable sections. The one or more detachable sections may each be coupled to conductive material, such as a filament, fiber, network or patch of carbon nanotubes (CNTs) or carbon nanofibers (CNFs). Each of the one or more detachable sections may be deployed by positioning the catheter at a desired position and securing the section utilizing a screw. In some embodiments, the deployment process may be repeated for additional detachable sections if desired.

In another embodiment, systems and methods for deploying and securing conductive materials to a region of tissue may utilize a catheter with an adjustable tip. Conductive materials may be coupled to one or more securing mechanisms and positioned within the catheter. In some embodiments, the securing mechanisms may be clips, anchors, alligator clips, anchors with barbs, or the like. To deploy the conductive materials, a securing mechanism may be positioned at or near the tip of the catheter, which is positioned at a tissue region of interest. In some embodiments, the securing mechanism may be deployed or secured by actuating the adjustable opening of the catheter. As nonlimiting examples, the opening may be expanded to release clips that will clip onto the tissue; or the catheter and/or securing mechanism may be advanced to insert an anchor into the tissue and/or the opening of the catheter may be reduced to cause barbs provide by the anchor to lock into tissue. In some embodiments, the deployment process may be repeated for additional clips or anchors if desired.

In yet another embodiment, a catheter may provide a needle. Conductive material may be provided in the form of a filament or fiber that is bundled, coiled, or spooled within the catheter and threaded through the hollow center of the needle. The needle may provide a stopping mechanism that prevents the filament or fiber from retracting back into the needle. The needle may be inserted into a tissue region of interest to drive the filament or fiber into the tissue, and when the needle is retracted, the filament or fiber remains in the tissue. In some embodiments, the deployment process may be repeated for additional regions of interest if desired.

The foregoing has outlined rather broadly various features of the present disclosure in order that the detailed description that follows may be better understood. Additional features and advantages of the disclosure will be described hereinafter.

### BRIEF DESCRIPTION OF THE DRAWINGS

For a more complete understanding of the present disclosure, and the advantages thereof, reference is now made to the following descriptions to be taken in conjunction with the accompanying drawings describing specific embodiments of the disclosure, wherein:

FIGS. 1A-1H are illustrative embodiments of a system for distributing conductive materials utilizing corkscrew-shaped screws;

FIG. 2 is an illustrative embodiment of an alligator clip coupled to conductive materials;

FIG. 3 is an illustrative embodiment of a catheter with an adjustable opening;

FIGS. 4A-4D are illustrative embodiments of deploying conductive materials to a region of tissue utilizing clips;

FIGS. 5A-5D are illustrative embodiments of deploying conductive materials to a region of tissue utilizing anchors;

FIGS. 6A-6D are an illustrative embodiment of needle for deploying conductive materials to a region of tissue; and

FIGS. 7A-7C are images of an experimental example of deployed conductive materials.

### DETAILED DESCRIPTION

Refer now to the drawings wherein depicted elements are not necessarily shown to scale and wherein like or similar elements are designated by the same reference numeral through the several views.

Referring to the drawings in general, it will be understood that the illustrations are for the purpose of describing particular implementations of the disclosure and are not intended to be limiting thereto. While most of the terms used herein will be recognizable to those of ordinary skill in the art, it should be understood that when not explicitly defined, terms should be interpreted as adopting a meaning presently accepted by those of ordinary skill in the art.

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

Existing treatment of heart-conduction pathologies, such as arrhythmias, is conducted through administration of drugs or controlled, localized deactivation of some areas of the heart (i.e., catheter ablation). However, these treatments have some drawbacks, such as variable efficacy in different patient populations and potentially dangerous chemical side effects. Further, ablation is irreversible, as it is predicated on additional deliberate scarring of the heart, and it does not directly address the mechanism of reentrant arrhythmias, namely impaired conduction. In other words, the areas of tissue that ablation is targeted at and utilized to remove are not problematic aside from the effect they have on conduction velocity. In contrast, delivery of a conductive material to an area of impaired conduction may address this mechanism without causing further damage to a tissue region (e.g., myocardium), and the treatment can be reversed by removal of the conductive material at a later time. Furthermore, these systems and methods discussed further herein are the first example of a catheter-based, intra-cardiac deployment of a conductive material to repair an impaired region of damaged tissue.

Systems and methods for attaching flexible, conductive material to tissue are discussed herein. In some embodiments, the flexible, conductive materials may be filaments, networks, or patches. The flexible conductive materials may be delivered to any tissue region with impaired or null electric conductivity to repair normal functionality of the tissue region. As a nonlimiting example, the flexible conductive materials may be delivered via a catheter to a region of impaired or null conduction of the myocardium (e.g. scar tissue) to improve or repair heart function.

In some embodiments, the flexible, conductive materials may be carbon nanotube (CNT) fibers (CNFs), fibers made of graphene or graphitic carbon fibers, flexible metallic wire or foil, conductive polymer thread, or the like. The flexible, conductive materials may form a fiber, filament, network, patch, or the like. The flexible conductive materials may be delivered into or onto a tissue region with impaired or null conduction, thereby improving or repairing normal electric conductivity of the region. In many cases, areas of slowed or null conduction (e.g., scar tissue) in the myocardium can lead to re-entrant arrhythmias and dangerously disrupt the normal function of the heart. In some embodiments, a flexible conductive material can be either sutured through, or placed over the surface of, a slow conduction area or scarred myocardium in order to restore electrical conduction. In some embodiments, a conductive CNT fiber may take the form of a single filament, a linear bundle of filaments, or a woven mat of filaments.

To be effective in some embodiments, it is desirable for the conductive material to be placed and held in contact with the zone of impaired conduction and also with the healthy, normally-conductive tissue on either side of the area of impaired conduction.

In some embodiments, the flexible, conductive materials may be attached to a desired region of tissue. In some embodiments, the attachments occur via catheter delivery. In some embodiments, the attachments are permanent. In some embodiments, the attachments are semi-permanent. In some embodiments, the tissue is a cardiac tissue. In some embodiments, the flexible, conductive materials may be coupled to securing mechanisms that aid temporary or permanent attachment to a tissue region. The securing mechanism may be a screw, clips, anchors, or the like. Nonlimiting examples of securing mechanisms may include corkscrew-shaped screws, alligator clips, or anchors with barbs. In some embodiments, the securing mechanisms may be formed of electrically conductive materials.

In some embodiments, the flexible, conductive materials may be delivered to a desired region via a flexible catheter. In some embodiments, the catheter may provide two or more detachable tips. In some embodiments, the catheter may provide deployment mechanisms that aid in deploying and/or securing the flexible, conductive material to a desired region. In some embodiments, the deployment mechanism may provide gearing to rotate a securing mechanism, such as a screw. In some embodiments, the deployment mechanism may provide an adjustable opening that allows the diameter of the opening to be adjusted, including decreasing and increasing the size of the opening, to aid deployment of the securing mechanism.

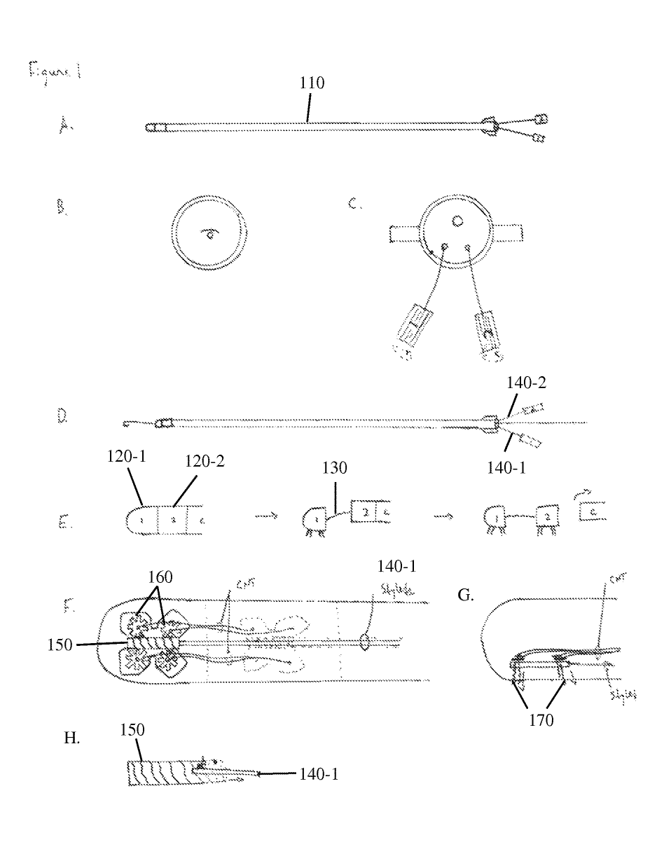

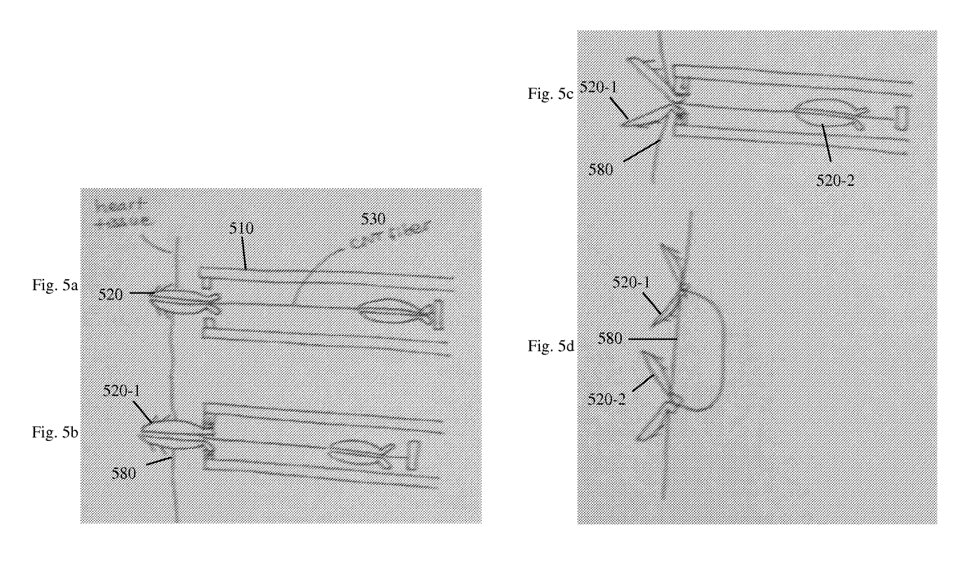

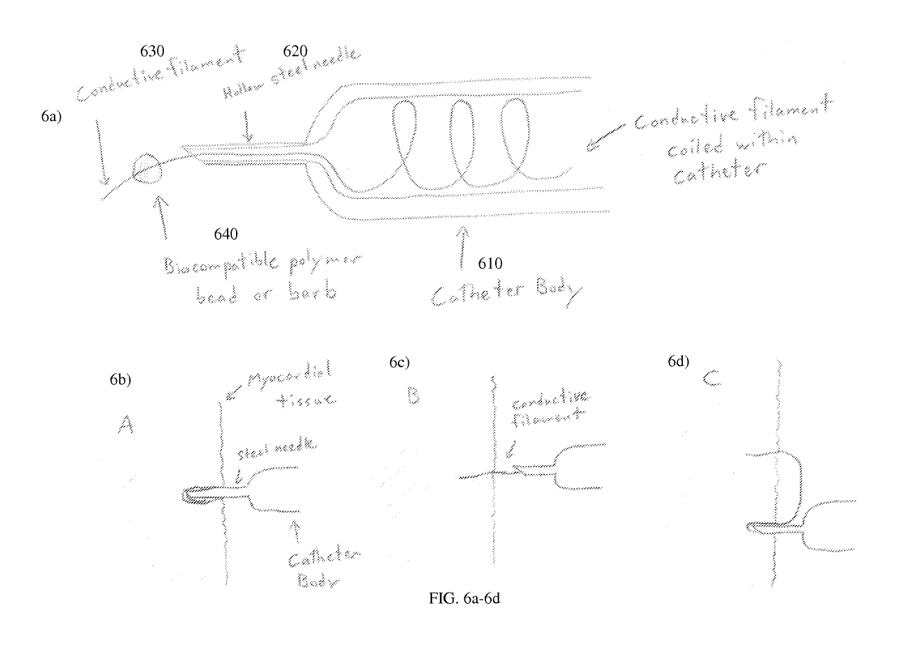

In some embodiments, a set of techniques can be used to deliver the flexible, conductive materials via a long, flexible catheter that can be inserted into the heart with a minimally invasive procedure. In some embodiments, the insertion of the catheter into the heart can be similar to the insertion methods used to insert pacemaker leads or ablation catheter tips into the heart during routine procedures. In some embodiments, the structure of the catheter can include a long, flexible catheter body made from plastic (such as nylon fiber) spanning 35 to 65 cm in length (FIG. 1A) and 5 mm in diameter (short axis, FIG. 1B-1C). In some embodiments, the catheter is not permanently inserted into the heart, but rather used to deposit the conductive materials and then be withdrawn. In some embodiments, the catheter can carry the conductive materials itself along with mechanisms (e.g. deployment/securing mechanisms) which allow the conductive materials to be attached stably to a desired region of tissue, such as the myocardium, near the target area and extended in a controlled way across the target area.

In some embodiments where the target area is a zone of null conduction (e.g., scar tissue), the conductive materials can also be attached stably to the tissue on the far side of the target area as well as in some cases to the tissue of the target area itself, all prior to removal of the catheter. In some embodiments, the conductive materials can be attached at opposite ends of a region null conduction or damaged tissue to bridge the damaged region and provide electric conductivity through the damaged region. In some embodiments, the mechanisms by which the conductive materials are attached to the tissue can ensure permanent or semi-permanent attachment. In some embodiments, the conductive materials are suitable for restoring conduction in regions of the myocardium with impaired conduction. This in turn can prevent dangerous arrhythmias.

In some embodiments, these systems and methods facilitates the treatment of cardiac arrhythmia by providing methods and devices for delivering conductive materials to damaged tissue regions to repair the region to provide normal heart function. In some embodiments, the methods and devices are minimally invasive and easily removable compared to more invasive, risky, or permanent treatments such as open heart surgery or cardiac ablation. In turn, the possibility of delivering this treatment in a minimally invasive way can allow the treatment to be applied to patients without creating undue risk of complications or long recovery times from surgery. This in turn can enable the treatment to be safer, cheaper, and more widely used.

In some embodiments, the methods and devices can also be used for attachment of conductive materials to any other organs or tissues of the body that are accessible to thin, flexible catheters. In some embodiments, suitable materials and methods may include patches to improve the transmission of electrical signals, patches for drug delivery, patches for biomedical sensing, or other uses.

In some embodiments, methods for deployment involve the insertion of the catheter into the heart and the attachment of a conductive material in multiple locations such that it bridges an area of impaired electric conductivity and remains permanently attached. In some embodiments, the methods includes a step of detachment of the conductive materials and any associated fastening device from the catheter itself, and the subsequent withdrawal of the catheter from the heart.

The insertion of the catheter into the heart may be similar to the insertion methods used to insert pacemaker leads or ablation catheter tips into the heart during routine procedures. In some embodiments, the catheter will not be permanently inserted into the heart, but rather will be used to deposit the conductive material and then withdrawn. The catheter may carry the conductive material itself along with securing/deployment mechanisms which allow the materials to be attached stably to the myocardium near the target area and extended in a controlled way across the target area, if necessary. If the target area is a zone of null conduction, the conductive material can also be attached stably to the tissue on the far side of the target area as well as in some cases to the tissue of the target area itself, all prior to removal of the catheter. In other embodiments, the attachment methods and mechanisms described herein can be used to attach ICD or pacemaker leads to the myocardium. The mechanisms by which the material is attached to the tissue will ensure permanent or semi-permanent attachment.

As a nonlimiting embodiment, highly conductive, flexible, microscale electrodes and suture thread were fabricated by using CNT fibers. The diameter of individual fibers can be varied between 8 and 200 μm. Thicker threads can be easily constructed by weaving together individual fibers. The electrodes and sutures do not show any degradation of the electric conductivity with kinking or bending. Because of their combination of electrical conductivity, mechanical strength, flexibility, fatigue resistance, and low contact impedance, these CNT fibers are an excellent material for functional electrodes for pacing and sensing heart electric activity. The fibers can also be used to restore cardiac conduction through electrically inactive cardiac scar tissue. In these applications, CNT fiber can be directly sutured on the heart tissue, either in external or intracardiac locations.

The methods described herein for catheter-based attachment of a conductive material to myocardial tissue or the like can be used in various applications, such as those described in PCT/US14/55893, filed on Sep. 16, 2014, which is incorporated by reference herein. The methods make possible to attach any thin and flexible conductive material to the myocardium as a part of a catheter-based procedure. Further, the methods may also be used as novel methods for attaching the end of an electrical lead into myocardium, such as in the implantation of pacemaker or implantable cardioverter-defibrillator (ICD) leads. In some embodiments, the material being implanted into the myocardium may be a conductive material, such as material composed of thin filaments which are implanted individually, one filament at a time, to form a conductive bridge over a region of conductive block. In some embodiments, the conductive material may also be in the form of a thin, flexible patch or a woven network of filaments used for the same purpose. In some embodiments, one or more of these methods may be used to assist in applying the treatment for ventricular arrhythmia which is described herein. The ability to apply the conducting material in question using a flexible catheter tool would mean that the treatment could be applied either to the inside or outside surface of the heart utilizing a minimally invasive procedure, requiring only a small incision through which the catheter can be inserted into the body or into a major blood vessel, and from there into the heart. The use of this method of delivery would have a significantly increased utility versus more invasive, risky, or permanent means of delivering treatment, such as open heart surgery or cardiac ablation. In turn, the possibility of delivering this treatment in a minimally invasive way will allow the treatment to be applied to patients without creating undue risk of complications or long recovery times from more invasive surgery, and thus will enable the treatment to be safer, cheaper, and more widely used. When used for attaching the ends of pacemaker or ICD leads to myocardial tissue, these methods would allow for a portion of the conducting material, which is typically encased in an insulated sheath, to extend a small distance out of the end of this insulating sheath and then to be embedded directly into the myocardial tissue, forming a good electrical contact. The various mechanisms described for use in these methods may each be incorporated into the design of pacemakers or ICD leads to provide a novel device. Many of the variations of methods described here will be of greatest use when the conducting material is somewhat flexible as discussed herein. The methods described here have increased utility because they allow for secure attachment to the heart wall in way that would be less mechanically disruptive and damaging to the tissue. Other uses that can be envisioned for these attachment methods are other medical treatments that require a flexible patch, or any other flexible material in the form of thin filaments or a woven network of thin filaments, to be delivered and attached to any other organs or tissues of the body that are accessible via thin, flexible catheters. This may include patches to improve the transmission of electrical signals, patches for drug delivery, patches for biomedical sensing, or other uses.

The methods and tools described here are the first to be described for catheter-delivered, intra-cardiac delivery of thread-like or patch-shape materials, such as conductive suture thread or a flexible conductive patch. Currently, catheter-based tools exist which can be used to place closed loops of suture that can be used to mechanically fasten, close, or constrict devices and tissue in the heart and on the heart surface. However, these methods are useful only for mechanical anchoring, and the tools that deliver suture in this case typically deliver only pre-formed loops of suture that may be tightened around a protruding structure. The methods and tools described here may be used to deliver a suture thread, flexible patch, or network to any arbitrary location on either the inner or outer surface of the heart. These methods and tools would be useful for delivering materials that function merely by maintaining electrical contact with the myocardial tissue. A novelty element of the systems and methods discussed herein lies in the description of how they may be used to connect a conductive material to multiple locations in such a way that electrical contact between the myocardial tissue and the conductive material spans a larger region of myocardial tissue (such as the area of a scar along with the healthy myocardial tissue around its borders). Further, variations of systems and method described herein represent methods of attachment for flexible, conductive materials that are less disruptive to tissue than the current state of the art, and therefore represent not only a means of delivering continuous lengths of conductive materials to a region of tissue, but also a novel means of attaching conductive materials for other devices, such as pacemaker or ICD leads, in way that causes less damage to tissue. The development of these methods and tools for catheter-based delivery of conductive material will allow novel treatments based on implantation of conductive elements to be carried out in a minimally invasive way, without the need for extensive surgeries to give physicians clearer access to the exterior or interior of the heart. This will make the use of such novel treatments far more feasible, shorter to deliver, less expensive, and safer for the health of the patient.

As discussed previously, the novel systems and methods may include a specialized catheter, or a specially designed pacemaker or ICD lead, capable of carrying out the delivery and permanent attachment of a conductive material to a desired tissue region. In broad terms, the process may involve the insertion of the catheter or lead into the heart by way of a major blood vessel and the attachment of the conductive material in one or more locations such that it either forms an electrical connection between the undamaged tissue and another device to which it is attached distally, or forms an electrical connection between two or more areas of undamaged tissue via multiple attachment points to bridge or span a damaged region of tissue (e.g., damaged myocardial tissue). If the goal is to form an electrical connection to a device, the catheter in question may be an end of a lead for the device, in which case the procedure would then be complete. If the goal is to form a conductive bridge between areas of myocardial tissue independent of a device, then the conductive material and any associated fastening device may be detached from the catheter itself, and the catheter would be withdrawn from the heart.

There are several variations on the methods and mechanisms that can be used to achieve inter-cardiac implantation of conductive element by a catheter tool as discussed further herein. As discussed previously, the conductive material may comprise a single, continuous, flexible thin filament or fiber. In some embodiments, the conductive materials will comprise a mesh of network of filaments woven together. In some cases this mesh may be woven in such a way as to impart “shape memory” which will provide an elastic property to the material. This allows stretching of the mesh. Upon release, a “recoil” will allow the mesh or any connecting ends or other locations made of similar materials to clasp onto the cardiac tissue, thereby providing increased stability to the placement of the conductive material by allowing it to remain in close contact with the myocardial surface, even at points of contact beyond those at which it is mechanically attached. This method may be used in combination with various embodiments of the attachment methods described herein or may be used independently. Further, either the entire mesh or only part of the mesh may have this “shape memory” property. In some embodiments, the conductive material will comprise a thin flexible patch or sheet of material. In the embodiments described above, the conductive material may be composed of metal wires, metal foil, conductive polymer, CNT fibers, or thin conductive CNT films.

FIGS. 1A-1G are an illustrative embodiment of a system and method for attaching conductive materials to a desired tissue region via catheter delivery. In one embodiment, a catheter 110 has a hollow lumen through which a 0.014 inch diameter wire may be inserted as a “rail” or guide (FIG. 1D). The tip of the catheter has one or more detachable components 120-1 and 120-2, detachable in stages, and each of these detachable components is fastened securely to an end of a continuous, flexible conducting material 130. For the purpose of clarity and simplicity, the conducting material 130 is illustrated as a filament or fiber of CNTs or CNFs coupled with two detachable components 120-1 and 120-2 in the example embodiments below. However, in other embodiments, there may be two or more detachable components and/or the conducting material may be a network or mesh of CNTs or any of the other types of conductive material as discussed previously.

This conducting material 130 spans the length from detachable stage 1 (tip 120-1) and stage 2 (middle 120-2) and forms the electrical “bridge” that will ultimately connect span a region of tissue (FIG. 1E), such as a damage portion of myocardium. In some embodiments, two or more detachable stages may be provided and utilized in a similar manner as described further herein. Stages 1 (tip 120-1) and 2 (middle 120-2) of the catheter will be attached to the catheter with two inner stylets (140-1 and 140-2). The first stylet 140-1 is labeled “1” and is responsible for deploying/detaching the tip. When the tip of the catheter is at the desired location and ready to be deployed, actuation or rotation of the stylet 140-1 is translated to a worm screw 150, which in turn, rotates internal gears 160. These internal gears 160 are attached to corkscrew-shaped screws 170 (FIG. 1F-1G). Thus, rotation of stylet 140-1 results in the screw 170 corkscrewing into myocardium and securing tip 120-1 to the tissue. While the embodiment shown provides four gears and four screws, other embodiments may provide any suitable number of gears and screws. In some embodiments, a single gear and screw may be provided. In other embodiments, two or more gears and two or more screws may be provided.

In some embodiments, the interface between stylet 140-1 and the tip worm screw is a square-in-peg design. Further, tip 120-1 may be detachable by removal of the stylet 140-1 from the catheter. In some embodiments, a tooth-latch may be pulled to free the stylet 140-1 from the worm screw 150 if greater stability is an issue (FIG. 1H). Conductive material 130 is pre-attached to detachable stage 1 (120-1) and detachable stage 2 (120-2), such that when stage 1 is attached to the myocardium and stage 2 is still attached to the catheter, this conductive material may be unwound or unfurled from within the body of the catheter and stretched to the desired location for stage 2 deployment (FIGS. 1E-1H). The conductive material 130 may have a pre-specified maximum extension length beyond which that particular catheter will not be rated to provide electrical bridging. After stage 1 (tip 120-1) is secured at a desired location, the catheter 120 may be moved to another desired location for stage 2 (middle 120-2) to be attached. Stage 2 (120-2) may be operated in the same manner as described for stage 1 (tip 120-1) to secure stage 2 to the desired location. For example, once the catheter is pulled back and stage 2 (120-1) is in the desired location, rotation of stylet 2 (not shown) will deploy corkscrews that will affix stage 2 to myocardium. Removal of stylet 2 may cause of detachment of stage 2 from the body of the catheter, and the conductive material 130 may form a bridge between the two locations at which stage 1 (tip 120-1) and stage 2 (middle 120-2) were deployed. After the electrical bridge is deployed, the catheter body and 0.014 inch rail may be removed from the heart, leaving only the bridge. In some embodiments, the securing mechanism (e.g. corkscrews from stages 1 and 2) may be made of a conductive material and may also be in electrical contact with the conductive bridge, such that their deployment will improve the electrical contact between the myocardium and the conductive bridge.

A variation of this embodiment comprises attaching portions of the conductive material directly to the tips of the corkscrews used for the implantation and fixation in the myocardium. In this variation, the act of driving the corkscrews into the myocardium will also embed part of the conductive material into the tissue itself, ensuring additional electrical and mechanical connection between the conductive material and the myocardium. Improved electrical connection may allow for better charge transfer between the myocardium and the conductive material and thus improved performance. Further, improved mechanical connection may reduce the risk of detachment of the conductive material from the myocardium or the securing mechanism, either during the implantation procedure or at a later time due to the movement of tissue or blood flow near the conductive material. As an additional variation, the corkscrew and the conductive material can be coated with a biodegradable adhesive material. This adhesive material would persist for the duration of the implantation surgery. Afterwards, if the conductive material has been driven into the myocardium along with the corkscrew, or if endothelialization has occurred to form a new layer of tissue over the implant, the adhesive would no longer be necessary to keep the conductive material attached to the corkscrews or the myocardium. Possible choices for adhesive material include, but are not limited to, polyethylene glycol (PEG), chitosan, sucrose solutions, and gelatin.

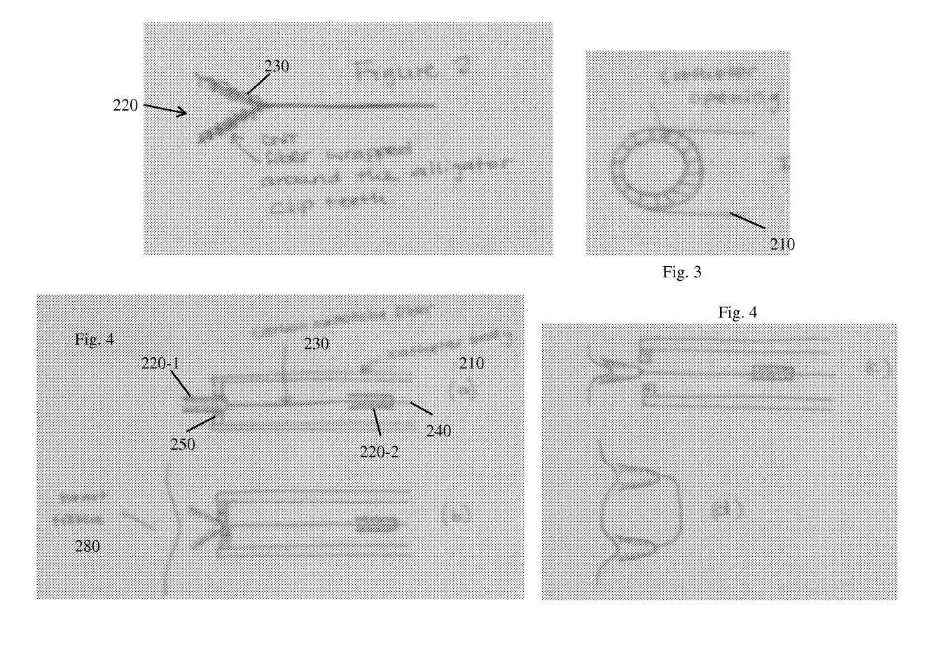

FIG. 2 is another illustrative embodiment of a system and method for attaching conductive materials to a desired tissue region via catheter delivery. In this embodiment, the securing mechanism may be two or more pairs of alligator clips, which take the place of stage 1 and stage 2 in the previously discussed embodiment and may be used to attach the ends of the conductive material to myocardial tissue. These alligator clip style securing mechanisms 220 may be utilized to attach the flexible, conductive material 230 to the myocardium in desired locations to bridge or span a region of tissue. For the purpose of clarity and simplicity, the conducting material 230 is illustrated as a filament or fiber with two alligator clips 220-1 and 220-2 in the example embodiments below. However, in other embodiments, there may be two or more clips and/or the conducting material may be a network or mesh of CNTs. As shown in FIG. 3, the catheter 210 is composed of a flexible body with an opening at the end. The opening of the catheter is adjustable to allow the diameter of the opening to be constricted or increased in size. Further, the adjustable catheter opening may be utilized to actuate the securing mechanism to attach to a desired tissue region.

FIGS. 4A-4D illustrate a deployment sequence for an illustrative embodiment utilizing alligator clips 220-1, 220-2. The catheter body 210 contains a hollow space through which a guide rod(s) 240 can be inserted. At the end of the guide rod 240 is at least one alligator clip 220-1 with one end of a continuous length of conductive material 230 attached to it. The conductive material 230 will extend from one alligator clip 220-1 to the other 220-2 within the catheter. The conductive material 230 can be permanently affixed to the alligator clips 220-1, 220-2. Further, conductive material 230 may be affixed in such a way that it will be pinched between the clips 220-1, 220-2 and myocardial tissue when the clips are closed. As a nonlimiting example, if the conducting material 230 is a filament or bundle of filaments, it may be wrapped around each of the jaws of the clips (FIG. 2). In some embodiments, the alligator clip 220-1, 220-2 jaws can be opened or closed by changing the size of the catheter body to actuate (i.e. pinch or release) ends of the clips as shown in FIGS. 4b-4c. The clips can be used to securely grab a portion of tissue, such as the myocardium, by increasing the diameter of the catheter to release the ends of the clips as shown in FIG. 4c. Further, the catheter 210 may be moved away from the clip 220-1 to move the clip out of the lumen of the catheter so that it remains fixed to the chosen area of attachment.

As a nonlimiting example of the process, the catheter 210 may be moved close to the first attachment area (FIG. 4a), and a guide 240 attached to the first alligator clip 220-1 is pushed to the very end of the catheter body 210. As the first alligator clip 220-1 is pushed outside the catheter body 210 near the first attachment, the opening 250 at the end of the catheter body may be constricted in order to squeeze the clip 220-1 and allow the jaws of the clip to open (FIG. 4b). The clip 220-1 is put in contact with a tissue region of interest 280, such as heart tissue, and the clip is allowed to close again by increasing the size of the opening 250 at the end of the catheter body, thereby securing the clip to the tissue (FIG. 4c). Then, the catheter body 210 is pushed back allowing the clip 220-1 to move away from the lumen of the catheter and the conductive material 230, which is still attached to the other alligator clip 220-2 within the lumen, to be strung out from the end of the catheter body. In some embodiments, the above described steps may be repeated for the second alligator clip 220-2 at another tissue region. For example, the catheter tip may be moved to a second tissue area of interest. The second alligator clip 220-2 may be advanced to the end of the catheter body using the guide 240, or another separate guide for the second alligator clip. Further, using the same steps described above for the first clip, the second alligator clip 220-2 may be attached to the second tissue area. The second clip 220-2 may then be released from the catheter 210, and the catheter may be withdrawn from the heart leaving the conductive material 230 and alligator clips 220-1, 220-2 behind in a proper deployment position as shown in FIG. 4d.

Another embodiment of a deployment/securing mechanism is shown in FIGS. 5a-5d. In some embodiments, the conductive material 530 may be coupled to anchors with barbs 520, such as suture anchors. The structure of an illustrative embodiment of suture anchors is described in U.S. Pat. No. 5,486,197. For the purpose of clarity and simplicity, the conducting material 530 is illustrated as a filament or fiber with two anchors 520 in the example embodiments below. However, in other embodiments, there may be two or more anchors and/or the conducting material may be a network or mesh of CNTs.

Each anchor 520 comprises an expandable barbed end that is initially collapsed into a sharp tip to aid ease of entry into tissue. The barb on the anchor 520 is designed so that it can easily penetrate tissue, such as the myocardium, while collapsed. However, when the anchor 520 is expanded or deployed to an open position, the anchor cannot easily be withdrawn while open. The catheter 510 may be similar to the previously described catheter with an adjustable opening. The catheter 510 contains a hollow space containing one or more guide wires for the anchors 520. For example, the guide wire(s) may be tipped with one or more anchor(s) 520, with each end of the conducting material 530 attached to an anchor within the catheter 510. Further, the catheter 510 may provide an adjustable opening that allows the diameter of the opening to be adjusted as desired to actuate the anchor(s) 520.

The collapsed tip of the first anchor 520-1 may be advanced to the end of the catheter, and the catheter 510 tip is moved near a first tissue region of interest 580, such as the myocardium. The tip of the first anchor 520-1 is pushed into the tissue region 580 by advancing the catheter 510 and/or guide wire until the anchor has properly penetrated the tissue. Once the anchor 520-1 is embedded, the tip of the catheter 510 is contracted to pinch the anchor open to expanding the anchor so that it latches onto the interior of the tissue with the barbs as shown in FIG. 5c. The first anchor 520-1 may then be detached from the guide wire and catheter 510, and the conductive material 530 is spooled out or unfurled from inside the catheter as the catheter tip is moved to a second tissue region of interest. The second anchor 520-2 may then be deployed in the same manner as discussed for the first anchor 520-1. Once deployment is complete, the catheter 510 may release the anchors and be subsequently withdrawn from the heart so that a conductive bridge comprising the anchors 520-1 and 520-2 and the conductive material 530 remains over a tissue region 580 as shown in FIG. 5d.

FIGS. 6a-6d are an illustrative embodiment of a needle arrangement for deploying or securing the conductive material to a tissue region of interest, such as the myocardium. In some embodiments, a catheter 610 may be tipped with a hollow needle 620, similar to a syringe needle or the like. The conductive material 630 may comprise a single continuous wire or filament, which is thin and flexible, and which is coiled or spooled within the catheter body close behind the needle tip. One end of the filament may be threaded through the hollow lumen of the needle. To prevent the portion of the filament that is past the tip of the needle from retracting back into the catheter body, a stopping mechanism 640 may be provided at or near the tip of the needle, such as a small barb, plastic or metal bead, knot, or the like. This stopping mechanism may be placed in such a way that a certain length of filament remains exposed.

As a nonlimiting example, a depiction of the embodiment in which the catheter 610 is tipped with a hollow steel needle 620, through which a flexible conductive material is threaded is shown. The filament 630 may be coiled within the body of the catheter 610 in such a fashion that it can be drawn out continuously through the hollow needle 620. An initial portion of the conductive filament 630 may be advanced past the end of the needle before use, and affixed with a bead or a barb 640 of biocompatible material (either metal or polymer) which will act as a stopper and prevent it from sliding back into the needle. FIGS. 6b-6d depicts the procedure by which this tool can be used to attach a single filament of conductive material 640 to several locations in a region of myocardial tissue. In FIG. 6b, the needle with a portion of protruding conductive material is inserted into the myocardium. In FIG. 6c, the needle is withdrawn, and the adhesive forces of the tissue closing around the exposed conductive filament hold it in place and a longer section of conductive filament is drawn forward through the needle as the catheter withdraws. In FIG. 6d, the catheter has been moved to another location in the myocardium, allowing the conductive filament to spool out further. The needle is re-inserted in this new location, and a section of exposed conductive filament is drawn into the tissue with the needle and will form the next anchoring point after the needle is withdrawn.

To deploy or secure the filament to a tissue region of interest, the catheter tip may be positioned near a first site desired for filament attachment, and the catheter is moved forward in such a way that the needle and exposed fiber are driven into the tissue. The filament size, flexibility, and the strength of the adhesion between the filament and the tissue are chosen in such a way that the needle can be withdrawn from the tissue without withdrawing the embedded length or portion of the filament as well. As the needle is withdrawn, a fixed length of filament should be drawn out through the hollow lumen of the needle from the space inside the catheter. Once a sufficient length of filament has been drawn out in this way, the process may be repeated at another location of the tissue. The process may be repeated several times until the filament has been embedded in a series of locations with a continuous and conductive length of filament running between these locations of attachment. The filament can therefore be embedded in a series of locations and in any suitable geometry chosen by the user of the tool to bridge a damaged region of tissue. This method would be suitable for creating a single implantation point, one line of continuous conductive filament, a network of crossing filaments to achieve a conductive bridge over a 2 dimensional area, or the like. In some embodiments, the filament may be fully or partially coated with a biodegradable adhesive to further increase adhesion to tissue, thereby making implantation easier and more stable for a desired length of time.

Any of the embodiments described above may utilize biodegradable, bio-absorbable materials for the construction of the deployment and/or securing mechanisms, such as the screws, clips, or anchors discussed previously. Exemplary biodegradable, bio-absorbable materials include, but are not limited to, biodegradable polymers such as polycaprolactone (PCL), polylactic acid (PLLA), or bio-absorbable metals such as iron and manganese alloys. In some embodiments, the biodegradable, bio-absorbable materials and deployment/securing mechanism design may be realized so that the degradation rate of the stage allows for the complete endothelialization of the conductive material (3-5 weeks). Once the deployment/securing mechanisms are dissolved by degradation or absorption, the conductive materials is completely and permanently encapsulated in the tissue and in good electrical contact with the target tissue region, such as myocardium. In such embodiments, the material used to construct the deployment/securing mechanism may also be conductive, to ensure continuous, good electrical conduction between the tissue and the implanted conductive materials.

Several of the embodiments described above detail methods for attaching a conductive material to tissue at two points. However, in other embodiments, these methods may be expanded upon by utilizing a catheter and/or deployment and securing mechanism with a greater number of anchoring mechanisms, such as two or more anchoring mechanisms. Further, some embodiments may utilize a network or patch of conductive materials in place of the filament. The anchoring mechanisms may each be attached to the conductive material at various different points in accordance with the conductive materials utilized. In the case of a filament or fiber of conductive materials, the anchoring mechanisms may be distributed along the length of the filament or fiber. In the case of a network or patch of conductive materials, the anchoring mechanisms may be distributed along the edges of the network or patch. Further, some embodiments may also provide additional anchoring mechanisms distributed in an interior region away from the edges of the network or patch to ensure the interior is in contact with tissue. The conductive material may be spread between a series of attachment points over a two-dimensional area of tissue, such as the myocardium. For example, the various anchoring mechanisms of a filament of conductive material may be arranged in a nonlinear manner to cover a two-dimensional area of tissue. In yet another example, the anchoring mechanisms for a network or patch of conductive materials may be spread out so that the network or patch covers a two-dimensional area of tissue. Alternatively, any of the methods described above could be applied to deploy and/or secure one end of a conductive material operating as a lead for an electronic device (e.g., pacemaker or ICD lead) to tissue.

### Experimental Example

The following examples are included to demonstrate particular aspects of the present disclosure. It should be appreciated by those of ordinary skill in the art that the methods described in the examples that follow merely represent illustrative embodiments of the disclosure. Those of ordinary skill in the art should, in light of the present disclosure, appreciate that many changes can be made in the specific embodiments described and still obtain a like or similar result without departing from the spirit and scope of the present disclosure.

### Example

### Attachment of CNT Fibers Through a Single-Point Tool

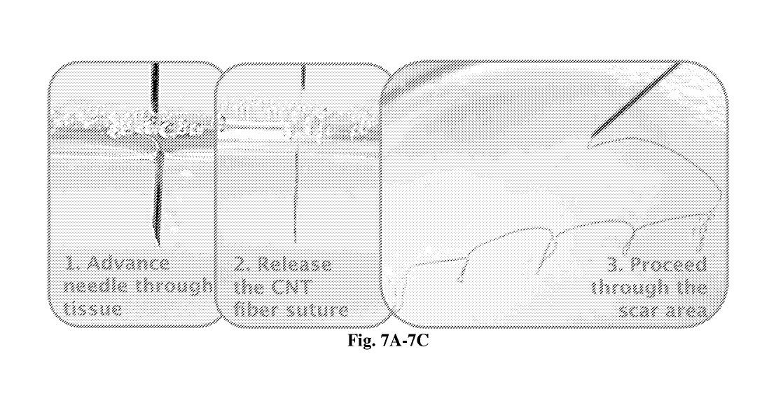

To investigate the feasibility of the CNT fiber implant method, a series of studies were performed in which the CNT fibers were serially imbedded within porcine myocardium using a single-point tool. The fibers were threaded through the lumen of a syringe needle and driven into the myocardial tissue. When the needle was retracted after being driven into the tissue, the fiber remained in place (i.e., it was implanted in the tissue) and a new section of fiber was drawn through the lumen, allowing the process to be repeated to form a series of “stitches.” The reason for this behavior is the high surface area of the CNT fibers, which allows adhesive forces on their surface to dominate once the wires are embedded in the myocardium. The CNT fibers remained stable, so they could be delivered contiguously in 9 locations during the same procedure. In these initial studies, the implants were stable enough to resist the flow of fluid across the surface, but the fibers could be removed by pulling on them. FIG. 7A-7B respectively show insertion and release of the CNT fiber in gelatin, a transparent model fluid mimicking mechanical properties of the myocardium. FIG. 7C shows distribution of the CNT fiber at several locations.

Embodiments described herein are included to demonstrate particular aspects of the present disclosure. It should be appreciated by those of skill in the art that the embodiments described herein merely represent exemplary embodiments of the disclosure. Those of ordinary skill in the art should, in light of the present disclosure, appreciate that many changes can be made in the specific embodiments described and still obtain a like or similar result without departing from the spirit and scope of the present disclosure. From the foregoing description, one of ordinary skill in the art can easily ascertain the essential characteristics of this disclosure, and without departing from the spirit and scope thereof, can make various changes and modifications to adapt the disclosure to various usages and conditions. The embodiments described hereinabove are meant to be illustrative only and should not be taken as limiting of the scope of the disclosure.

What is claimed is:

1. A system for securing conductive material to tissue, the system comprising:
a catheter;
a first tip secured to an end of the catheter, wherein the first tip is detachable from the catheter;
a conductive material positioned within a lumen of the catheter, wherein the conductive material is coupled to the first tip, and the conductive material comprises carbon nanotubes or carbon nanofibers; and
at least one screw coupled to the conductive material, wherein the screw is actuated to secure the first tip and the conductive material to a first region of tissue.

2. The system of claim 1, where in the conductive material is a fiber, filament, network, or patch of the carbon nanotubes or carbon nanofibers.

3. The system of claim 1, further comprising:
a second tip coupled to the catheter, wherein the second tip is detachable from the catheter, and the second tip is coupled to the conductive material at a location different from the first tip; and
a second set of screws coupled to the conductive material at a location different from the at least one screw, wherein the second set of screws are actuated to secure the second tip and the conductive material to a second region of the tissue.

4. The system of claim 1, further comprising:
a stylet positioned within the catheter;
a first gear coupled to the stylet; and
at least one second gear coupled to the at least one screw, wherein the at least one screw is mated to the first gear, and the first gear is actuated to rotate the at least one screw.

5. The system of claim 4, wherein the stylet is detachable from the first gear.

6. A method for securing conductive material to tissue, the method comprises:
positioning a first tip of a catheter near a first region of tissue;
actuating a first screw coupled to a conductive material positioned within a lumen of the catheter, wherein the conductive material comprises carbon nanotubes or carbon nanofibers, and actuation of the first screw secures it to the first region; and
detaching the first tip of the catheter from the catheter, wherein the first tip and the conductive material are secured to the first region by the first screw.

7. The method of claim 6, where in the conductive material is a fiber, filament, network, or patch of the carbon nanotubes or carbon nanofibers.

8. The method of claim 6, wherein the first screw is coupled to a stylet, and the stylet is rotated to actuate the first screw.

9. The method of claim 6, further comprising:
moving the catheter to position a second tip near a second region of the tissue;
actuating a second screw coupled to the conductive material, wherein actuation of the second screw secures it to the second region; and
detaching the second tip of the catheter from the catheter, wherein the second tip is coupled to the second screw, the second tip and the conductive material are secured to the second region by the second screw, and the conductive material bridges the tissue from the first region to the second region.

10. A system for securing conductive material to tissue, the system comprising:
a catheter providing an adjustable opening, wherein the adjustable opening allows a diameter of a catheter opening to be increased or decreased;
a conductive material positioned within a lumen of the catheter, wherein the conductive material comprises carbon nanotubes or carbon nanofibers; and
a first securing mechanism coupled to the conductive material, wherein the first securing mechanism is actuated by the adjustable opening to secure the first securing mechanism to a desired region of tissue.

11. The system of claim 10, where in the conductive material is a fiber, filament, network, or patch of the carbon nanotubes or carbon nanofibers.

12. The system of claim 10, wherein the first securing mechanism is a clip, anchor, alligator clip, or anchor with barbs.

13. The system of claim 10, wherein the diameter of the adjustable opening is increased to secure the first securing mechanism to the desired region of tissue.

14. The system of claim 10, wherein the diameter of the adjustable opening is decreased to secure the first securing mechanism to the desired region of tissue.

15. The system of claim 10, further comprising a second securing mechanism coupled to the conductive material at a location different from the first securing mechanism, wherein the second securing mechanism is actuated by the adjustable opening to secure the second securing mechanism to another region of the tissue.

16. A method for securing conductive material to tissue, the method comprising:
positioning a catheter near a first region of tissue, wherein the catheter providing an adjustable opening that allows a diameter of a catheter opening to be increased or decreased, and the catheter provides a first securing mechanism coupled to a conductive material, wherein the conductive material comprises carbon nanotubes or carbon nanofibers; and
actuating the first securing mechanism positioned within a lumen of the catheter near a tip of the catheter, wherein the first securing mechanism is actuated by the adjustable opening to secure the first securing mechanism to the first region of tissue.

17. The method of claim 16, where in the conductive material is a fiber, filament, network, or patch of the carbon nanotubes or carbon nanofibers.

18. The method of claim 16, wherein the first securing mechanism is a clip, anchor, alligator clip, or anchor with barbs.

19. The method of claim 16, wherein the diameter of the adjustable opening is increased to secure the first securing mechanism to the first region of tissue.

20. The method of claim 16, further comprising the step of advancing the first securing mechanism to penetrate the first region of tissue prior to actuating the first securing mechanism.

21. The method of claim 20, wherein the diameter of the adjustable opening is decreased to secure the first securing mechanism to the first region of tissue.

22. The method of claim 16, further comprising the steps of:
positioning the catheter near a second region of the tissue, wherein the catheter provides a second securing mechanism coupled to the conductive material at a location different from the first securing mechanism; and
actuating the second securing mechanism positioned within the lumen of the catheter near the tip of the catheter, wherein the second securing mechanism is actuated by the adjustable opening to secure the second securing mechanism to the second region.

23. A system for securing conductive material to tissue, the system comprising:
a catheter;
a needle positioned on a tip of the catheter;
conductive material provided within a lumen of the catheter and threaded through a tip of the needle, wherein the conductive material comprises carbon nanotubes or carbon nanofibers in the shape of a fiber or filament; and
a stopping mechanism positioned on the needle, wherein the stopping mechanism prevents the fiber or filament from retracting back into the catheter.

23. The system of claim 1, wherein the conductive material is coated with a biodegradable adhesive.

24. A method for securing conductive material to tissue, the method comprising:
threading a conductive material through a catheter and a needle positioned on a tip of the catheter, wherein the conductive material comprises carbon nanotubes or carbon nanofibers in the shape of a fiber or filament;
inserting the needle into a first region of tissue, wherein the needle embeds a first portion of the conductive material in the tissue, and a stopping mechanism that prevents the fiber or filament from retracting back into the catheter; and
withdrawing the needle from the first region of tissue, wherein the first portion of conductive material embedded in the tissue remains embedded in the tissue after withdrawal of the needle.

25. The method of claim 24, further comprising:
inserting the needle into a second region of tissue, wherein the needle embeds a second portion of the conductive material in the tissue; and
withdrawing the needle from the second region of tissue, wherein the second portion of conductive material embedded in the tissue remains embedded in the tissue after withdrawal of the needle, and the conductive material bridges the tissue from the first region to the second region.

26. The method of claim 24, wherein the conductive material is coated with a biodegradable adhesive.
